(19) United States
(12) Patent Application Publication
(10) Pub. No.: US 2025/0295438 A1
(43) Pub. Date: Sep. 25, 2025

(54) SYSTEMS AND METHODS FOR A SPINAL IMPLANT

(71) Applicant: Dignity Health, San Francisco, CA (US)

(72) Inventor: Michael Bohl, San Francisco, CA (US)

(21) Appl. No.: 19/229,835

(22) Filed: Jun. 5, 2025

Related U.S. Application Data
(63) Continuation of application No. 17/931,991, filed on Sep. 14, 2022.
Application No. 17/931,991 is a continuation of application No. 16/607,303, filed on Oct. 22, 2019, now abandoned.
(60) Provisional application No. 62/491,199, filed on Apr. 27, 2017.

(51) Int. Cl. A61B 17/70; A61F 2/30; A61F 2/44

(52) U.S. Cl. CPC A61B 17/7071; A61B 17/7049; A61B 17/7064; A61B 17/7067; A61F 2/30771; A61F 2/4405; A61F 2002/30172; A61F 2002/30785

(57) ABSTRACT

A spinal implant is disclosed for engagement to portions of a spinal segment after a laminectomy procedure. The spinal implant includes a first member and a second member in orthogonal relation relative to the first member such that the spinal implant generally defines a t-shape configuration. The spinal implant is formed using biocompatible materials to safely adapt to the surgical area. The spinal implant may include hooks or apertures for engaging with portions of bodily tissue. The spinal implant may be engaged to other spinal implants along the spine using one or more arms of an interconnecting arrangement.

This application is a continuation of U.S. patent application Ser. No. 17/931,991 filed on Sep. 14, 2022, which is a continuation application of U.S. patent application Ser. No. 16/607,303 filed on Oct. 22, 2019, now abandoned, which claims benefit of International Application No. PCT/US2018/029971 filed Apr. 27, 2018, which claims benefit of U.S. Provisional Application Ser. No. 62/491,199 filed Apr. 27, 2017, all of which are herein incorporated by reference in their entireties.

### FIELD

The present disclosure generally relates to the field of prosthetic medical devices and methods. Specifically, the present disclosure includes a spinal implant that may be positioned along an individual's vertebrae to simulate the natural spinous process and lamina portions of the vertebral arch removed during a laminectomy procedure.

### BACKGROUND

A laminectomy is a surgical procedure for removal of the vertebral arch, including the spinous process and portions of the lamina, located in the cervical, thoracic, lumbar, and sacral regions of the spine. This procedure may be performed on patients with back pain due to compression along the spinal cord or nerves, which may be caused from various spine diseases, including (but not limited to) degenerative, infectious, neoplastic, traumatic, and congenital pathologies. Removal of the vertebral arch allows for decompression of the spinal canal, and gives the surgeon access to the contents of the spinal canal as needed.

Despite its advantages, a laminectomy procedure may present various complications. For example, a laminectomy procedure inherently results in postoperative dead space around the surgical area. This dead space may lead to dangerous postoperative fluid collections, such as hematomas, and may lead to infection. Known methods to address these complications may involve rotating muscles in the patient's back to re-occupy the dead space. However, this generally requires a plastic surgeon, additional time, and may cause additional blood loss.

Further, some patients may require the application of a spinal fixation construct, which may include e.g., pedicle screws and rods, along a spinal segment for treatment of spinal instability, in addition to laminectomies for decompression of the neural elements. Any spinal fusion procedure carries the risk of causing adjacent segment disease or proximal junctional kyphosis. Moreover, such fixation constructs generally exacerbate the problem of postoperative dead space in the laminectomy defect because it is more difficult to bring the paraspinal muscles back towards the midline to fill this dead space after the pedicle screws and rods have been placed.

It is with these observations in mind, among others, that various aspects of the present disclosure were conceived and developed.

### BRIEF DESCRIPTION OF THE DRAWINGS

FIG. 1A is a postoperative posterior view of a spinal segment after a laminectomy procedure. The dotted lines indicate possible positioning of a spinal implant along postoperative dead space and cut portions of the spinal segment resulting from the laminectomy.

FIG. 1B is a postoperative posterior view of the spinal segment of FIG. 1A with a first embodiment of a spinal implant positioned along the spinal segment in the manner indicated by the dotted lines of FIG. 1A.

FIG. 2 is a perspective anatomical view of the first embodiment of a spinal implant of FIG. 1B.

FIG. 3 is a front view of the first embodiment of a spinal implant of FIG. 1B.

FIG. 4 is a postoperative posterior view of a spinal segment with a second embodiment of a spinal implant positioned along the spinal segment in a manner similar to FIG. 1B in order to at least partially occupy dead space formed subsequent to a laminectomy and spinal fixation procedure.

FIG. 5A is a perspective anatomical view of the second embodiment of a spinal implant positioned along a spinal segment and a spinal fixation construct including pedicle screws and rods.

FIG. 5B is a perspective anatomical view of the second embodiment of a spinal implant positioned along a spinal segment and a spinal fixation construct with optional rings formed around portions of the spinal fixation construct to maintain the spinal implant in a fixed position relative to the spinal segment.

FIG. 6 is a front view of the second embodiment of a spinal implant indicated in FIG. 5A positioned along the spinal segment and the spinal fixation construct.

FIG. 7 is a posterior view of a third embodiment of a spinal implant with a pair of arms aligned along opposing sides of the spinal implant and configured for connection with other arms of adjacent spinal implants or to the spinous processes of the superior and/or inferior spinal levels.

FIG. 8 is a perspective view of the spinal implant of FIG. 7 with arms configured for connections to adjacent implants or to the spinous processes of the superior and/or inferior spinal levels.

FIG. 9A is a perspective view of a fourth embodiment of a spinal implant for deployment along a spinal segment after a laminectomy procedure or for other applications.

FIG. 9B is a front view of the fourth embodiment of a spinal implant for deployment along a spinal segment after a laminectomy procedure or for other applications.

FIG. 9C is a perspective view of the fourth embodiment of a spinal implant with optional hooks for engaging other spinal implants and/or bodily tissue and a mounting rack for engagement with a track or spinal fixation hardware, as described herein.

FIG. 10 is a postoperative posterior view of a spinal segment with a fifth embodiment of a spinal implant positioned along the spinal segment in a manner similar to FIG. 1B in order to at least partially occupy dead space formed subsequent to a laminectomy and spinal fixation procedure.

FIG. 11 is a perspective view of the fifth embodiment of a spinal implant of FIG. 10 suitable for deployment along a spinal segment after a laminectomy procedure and spinal fixation procedure or for other applications.

Corresponding reference characters indicate corresponding elements among the view of the drawings. The headings used in the figures do not limit the scope of the claims.

### DETAILED DESCRIPTION

Aspects of the present disclosure relate to systems and methods for a spinal implant that are suitable for addressing various postoperative spine complications, among other advantages. The spinal implant may generally comprise a biocompatible body defining a first member resembling portions of the vertebral lamina, and a second member resembling a spinous process. The spinal implant may be configured for mounting directly to or proximate to bone tissue along a spinal segment subsequent to a laminectomy procedure, or may be engaged to the spinal segment together with a spinal fixation construct.

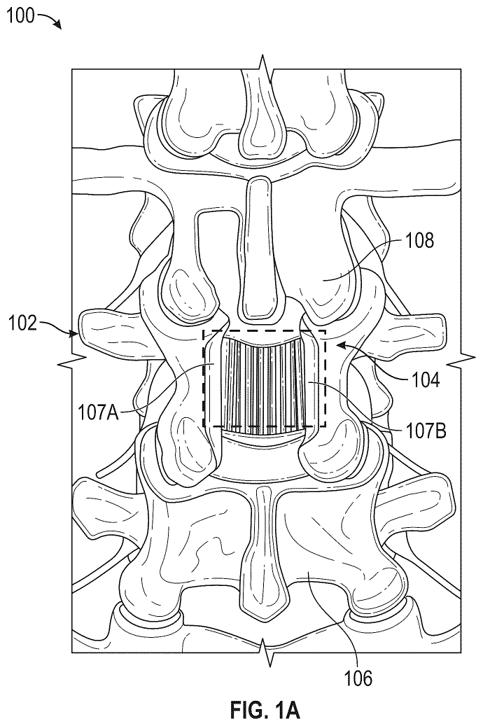

In some embodiments, the spinal implant may include one or more apertures or engagement members (e.g., hooks, or rings) for engaging with bodily tissue, as described herein. In addition, multiple spinal implants may be deployed along a human spine to accommodate the removal of more than one vertebral arch (multiple laminectomies), and the multiple spinal implants may be serially aligned along the spine, chained together or otherwise interconnected to increase spinal stability, among other features. Referring to the drawings, embodiments of a spinal implant are illustrated and generally indicated as 110, 210, 310, 410, and 510 in FIGS. 1B-11.

Referring to FIG. 1A, a system 100 is shown for purposes of describing one possible application of a spinal implant as described herein. The system 100 may generally include a spine 102 defining a first vertebral segment 104, a second vertebral segment 106, and a third vertebral segment 108, such that the first vertebral segment 104 is positioned between the second vertebral segment 106 and the third vertebral segment 108. In the system 100 depicted, a vertebral arch (not shown) associated with the first vertebral segment 104 has been removed as part of a laminectomy procedure to expose cut lamina portions 107A and 107B along the first vertebral segment 104. The dotted lines indicate possible positioning of a spinal implant along dead space and the cut lamina portions 107A and 107B of the first vertebral segment 104 resulting from the laminectomy.

Referring to FIG. 1B, a first embodiment of a spinal implant 110 according to the system 100 may be positioned along the first vertebral segment 104 of the spine 102 between the second vertebral segment 106 and the third vertebral segment 108 in the manner indicated, and may be generally mounted to or at least positioned along the cut lamina portions 107A and 107B or other portions of the first vertebral segment 104 in order to occupy the dead space resulting from the laminectomy procedure of FIG. 1A, and provide various other advantages as described herein.

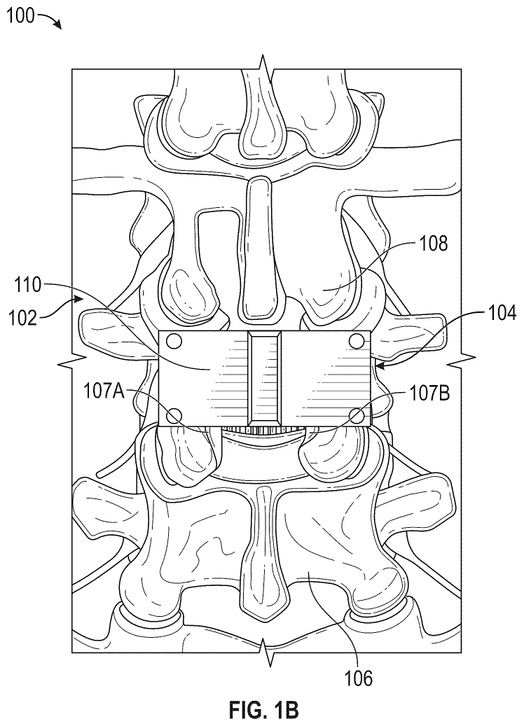

Referring to FIG. 2, the spinal implant 110 of FIG. 1B is shown, oriented along the first vertebral segment 104 between the second vertebral segment 106 and the third vertebral segment 108. As indicated, the spinal implant 110 may include a body 112 defining a first member 114. The first member 114 may be generally formed with dimensions suitable for engagement to portions of the first vertebral segment 104 such as the cut lamina portions 107A/107B, and the first member 114 may be generally rectangular in shape, and may be substantially planar as shown. The first member 114 of the body 112 may define a first lateral side 116, a second lateral side 118 opposite the first lateral side 116, a third side 120 adjacent the first lateral side 116 and the second lateral side 118, and a fourth side 122 opposite the third side 120, such that the third side 120 is oriented along the first vertebral segment 104. In some embodiments, the first lateral side 116 and the second lateral side 118 of the first member 114 may be formed with dimensions suitable for alignment with adjacent transverse process portions of the second vertebral segment 106 and the third vertebral segment 108.

In addition, the body 112 of the spinal implant 110 may further include a second member 130 oriented in perpendicular relation relative to the first member 114. The second member 130 may be generally rectangular as shown and may define a first end 132 in communication with the first member 114 along a generally central area of the fourth side 122 of the first member 114, and a second (free) end 134 opposite the first end 132, as shown. In some embodiments, the first member 114 and the second member 130 collectively define a general t-shape configuration that is intended to simulate the removed portions of a vertebral arch (not shown) along the first vertebral segment 104 subsequent to a laminectomy procedure. It should be appreciated that the body 112 of the spinal implant 110 is not limited to certain shapes and may take on various forms and dimensions so long as the spinal implant 110 accommodates the occupation of postoperative dead space, and provides other features as described herein. As further shown, one or more screws 140, pins, or other such fastening members, or adhesive (not shown) may be employed along the first member 114 to mount the first member 114 to portions of the first vertebral segment 104. In other embodiments, however, the spinal implant 110 may simply be positioned along the spine 102 as shown without screws 140, and the spinal implant 110 may be engaged with bodily tissue or otherwise held in place relative to the spine 102 without being directly mounted to the second vertebral segment 106.

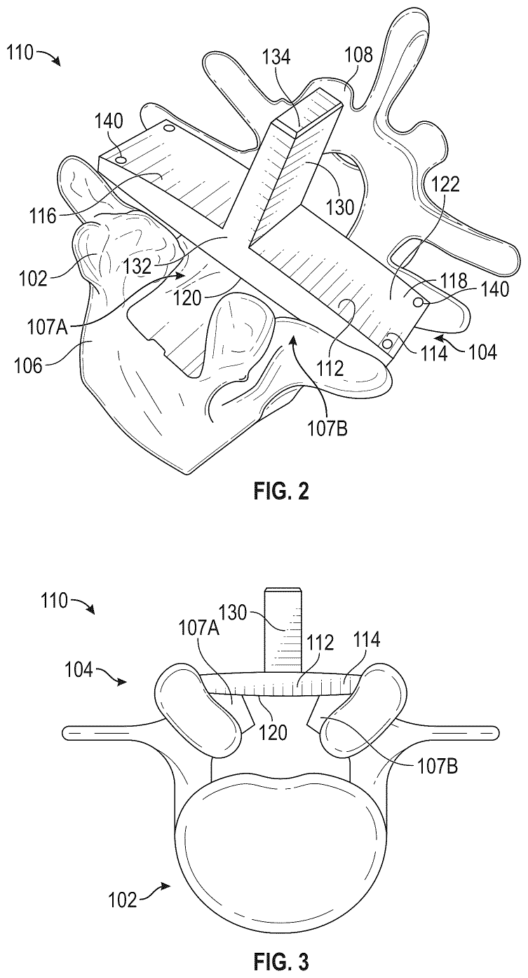

FIG. 3 illustrates additional detail regarding the possible engagement of the spinal implant 110 to the first vertebral segment 104 of the spine 102. As indicated, the third side 120 of the first member 114 of the body 112 may engage with or may be coupled to the cut lamina portions 107A and 107B, to position the second member 130 of the body 112 along the first vertebral segment 104 and the spine 102 as shown. In some embodiments, the first member 114 may be reduced in size (not shown) to fit between the cut lamina portions 107A and 107B, and/or may otherwise be shaped to occupy additional space along the first vertebral segment 104 as desired. In either case, the spinal implant 110 replaces at least some of the lost bone tissue resulting from the removal of the vertebral arch (not shown) of the laminectomy procedure applied to the first vertebral segment 104 in FIG. 1A.

The body 112 may be formed of various materials suitable for engagement around a surgical area and the spine 102. In some embodiments, the body 112 of the spinal implant 110 may be formed using one or more biocompatible materials for forming synthetic bone tissue, such as bone graft substitutes, or bone cement, and may include calcium sulfate and/or calcium phosphate to form the body 112 with synthetic bone-like properties. Utilizing such materials in these embodiments, the body 112 as formed may provide an interconnected, porous scaffold (not shown) that may allow bone tissue to grow safely within, through, and around the body 112 of the spinal implant 110. The biocompatible materials of the body 112 may further include antibiotics which may be absorbed by bodily tissue around the surgical area over extended periods of time. To form the body 112 in these embodiments, the biocompatible materials may generally be in the form of a powder, which may be received within one or more molds (pre-configured to desired shapes for the spinal implant 110) by injection or otherwise and mixed with one or more solutions to ultimately create synthetic bone tissue of a predefined configuration defined by the molds. In other embodiments, the body 112 may include metals, combinations of metals and synthetic bone tissue, or may be formed entirely of a metal material (e.g., molded, cast, extruded, or otherwise formed into the desired shape).

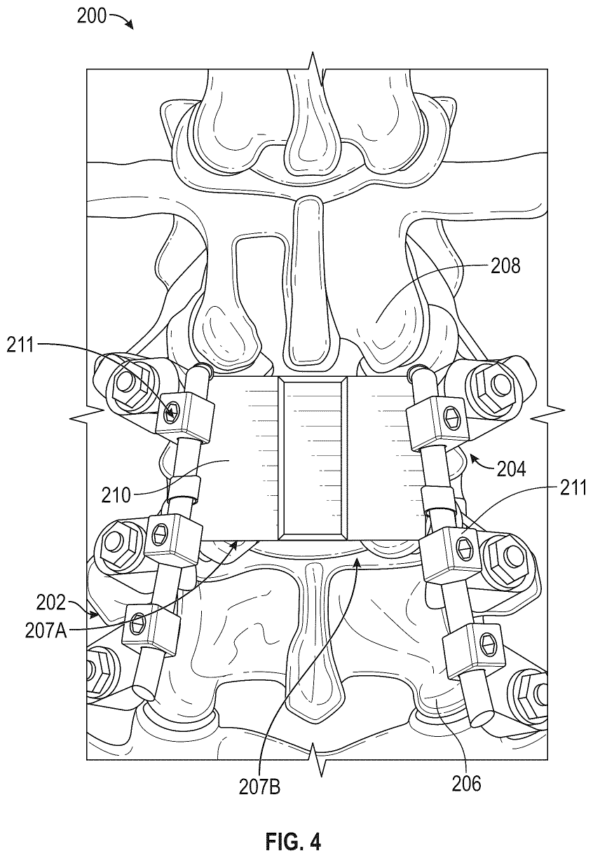

Referring to FIG. 4, a second embodiment of a spinal implant 210 according to a system 200, similar to the spinal implant 110, may be positioned along a first vertebral segment 204 of a spine 202 between a second vertebral segment 206 and a third vertebral segment 208 in the manner indicated, and may generally be mounted to or at least oriented along cut lamina portions 207A and 207B of the first vertebral segment 204 in order to occupy dead space resulting from a laminectomy procedure applied to the first vertebral segment 204. The spinal implant 210 may provide various postoperative advantages as described herein.

As further shown, the spinal implant 210 may be integrated with a spinal fixation construct 211 comprising one or more pedicle screws, rods, or other spinal fixation hardware that is coupled to the spine 202. In the embodiment shown, the spinal fixation construct 211 may include any cross link system for spinal fixation as would be understood by one skilled in the art. The spinal implant 210 may be engaged along the first vertebral segment 204 prior to application of the spinal fixation construct 211, together with application of the spinal fixation construct 211, or otherwise, as further described herein.

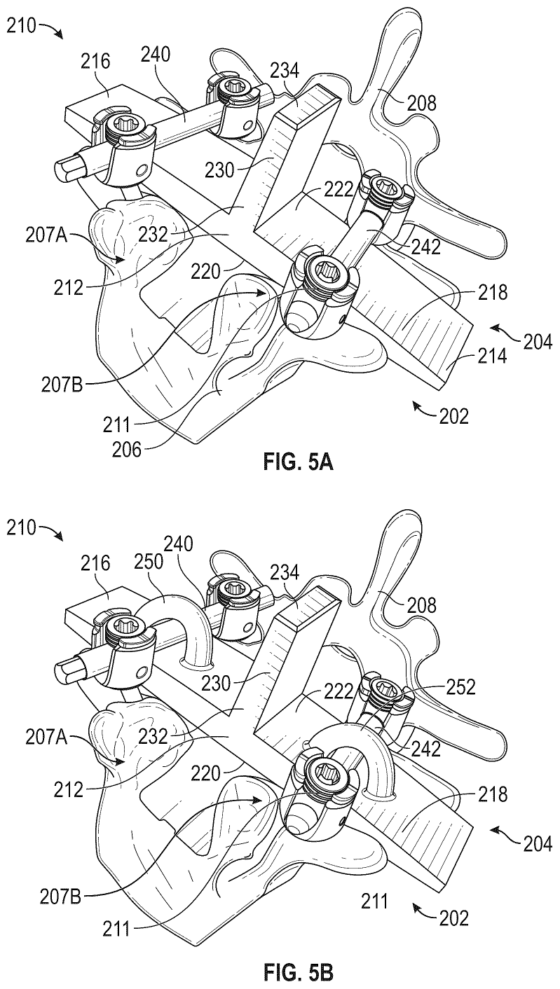

Referring to FIG. 5A, the spinal implant 210 of FIG. 4 is shown, oriented along the first vertebral segment 204 between the second vertebral segment 206 and the third vertebral segment 208. As indicated, the spinal implant 210 may include a body 212 defining a first member 214. The first member 214 may be generally formed with dimensions suitable for engagement to portions of the first vertebral segment 204 such as the cut lamina portions 207A/207B, may be generally rectangular in shape, and may be substantially planar as shown. The first member 214 of the body 212 may define a first lateral side 216, a second lateral side 218 opposite the first lateral side 216, a third side 220 adjacent the first lateral side 216 and the second lateral side 218, and a fourth side 222 opposite the third side 220, such that the third side 220 is oriented along and generally adjacent to the first vertebral segment 204. In some embodiments, the first lateral side 216 and the second lateral side 218 of the first member 214 may be formed with dimensions suitable for alignment with adjacent transverse process portions of the second vertebral segment 206 and the third vertebral segment 208.

In addition, the body 212 of the spinal implant 210 may further include a second member 230 oriented in substantially perpendicular relation relative to the first member 214. The second member 230 may be generally rectangular as shown and may define a first end 232 in communication with the first member 214 along a generally central area of the fourth side 222 of the first member 214, and a second (free) end 234 opposite the first end 232, as shown. In some embodiments, the first member 214 and the second member 230 collectively define a general t-shape configuration that is intended to simulate the removed portions of a vertebral arch (not shown) along the first vertebral segment 204 subsequent to a laminectomy procedure. It should be appreciated that the body 212 of the spinal implant 210 is not limited to certain shapes and may take on various forms and dimensions so long as the spinal implant 210 accommodates the occupation of postoperative dead space, and provides other features as described herein. One or more screws, pins, or other such fastening members, or adhesive (not shown) may be employed along the first member 214 to mount the first member 214 to portions of the first vertebral segment 204.

As further shown in FIG. 5A, the spinal implant 210 may be integrated with the spinal fixation construct 211. Specifically, at least a portion of the first lateral side 216 of the body 212 may be positioned below a first rod 240 of the spinal fixation construct 211; and at least a portion of the second lateral side 218 may be positioned below a second rod 242 of the spinal fixation construct 211 as shown. In this manner, the combination of the spinal fixation construct 211 and the spinal implant 210 occupies much of the dead space resulting from a laminectomy procedure applied to the first vertebral segment 104, and the spinal fixation construct 211 may assist to secure the spinal implant 210 in place as indicated. FIG. 5B illustrates one possible sub feature where the body 212 may include engagement members 250 and 252, which may define rings, hooks, or other suitable shapes, that may wrap at least partially around the first rod 240 and the second rod 242 and/or may receive at least a portion of the first rod 240 and the second rod 242 respectively, respectively for added support.

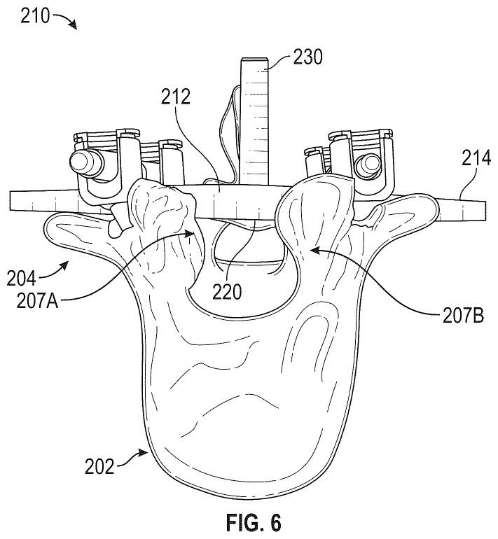

FIG. 6 illustrates additional detail regarding the possible engagement of the spinal implant 210 to the first vertebral segment 204 of the spine 202. As indicated, the third side 220 of the first member 214 of the body 212 may be coupled to and/or engaged with the cut lamina portions 207A and 207B, to position the second member 230 of the body 212 along the first vertebral segment 204 and the spine 202 as shown. In some embodiments, the first member 214 may be reduced in size (not shown) to fit between the cut lamina portions 207A and 207B, and/or may otherwise be shaped to occupy additional space around the first vertebral segment 204 as desired. In either case, the spinal implant 210 replaces at least some of the bone tissue resulting from the removal of the vertebral arch (not shown) of the laminectomy procedure applied to the first vertebral segment 204.

Similar to the spinal implant 110, the body 212 of the spinal implant 210 may be formed of various materials suitable for engagement around a surgical area and the spine 202. In some embodiments, the body 212 of the spinal implant 210 may be formed using one or more biocompatible materials for forming synthetic bone tissue, such as bone graft substitutes, or bone cement, and may include calcium sulfate and/or calcium phosphate to form the body 212 with synthetic bone-like properties. Utilizing such materials in these embodiments, the body 212 as formed may provide an interconnected, porous scaffold (not shown) that may allow bone tissue to grow safely within, through, and around the body 212 of the spinal implant 210. The biocompatible materials of the body 212 may further include antibiotics which may be absorbed by bodily tissue around the surgical area over extended periods of time. To form the body 212 in these embodiments, the biocompatible materials may generally be in the form of a powder, which may be received within one or more molds by injection or otherwise and mixed with one or more solutions to ultimately create synthetic bone tissue of a predefined configuration defined by the molds. In other embodiments, the body 212 may include metals, combinations of metals and synthetic bone tissue, or may be formed entirely of a metal material, to compliment metal-based embodiments of the spinal fixation construct 211.

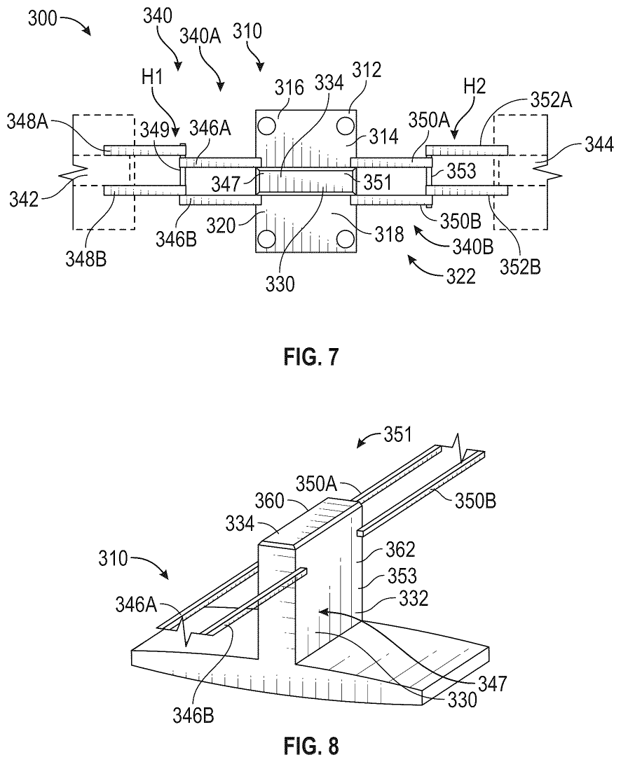

Referring to FIG. 7, a system 300 is shown for illustrating a third embodiment of a spinal implant 310 configured to accommodate multiple laminectomy procedures, or other applications. As indicated, the spinal implant 310 may include a body 312 defining a first member 314. The first member 314 may be generally formed with dimensions suitable for engagement to portions of a vertebral segment subsequent to a laminectomy procedure, may be generally rectangular in shape, and may be substantially planar as shown. The first member 314 of the body 312 may define a first lateral side 316, a second lateral side 318 opposite the first lateral side 316, a third side 320 adjacent the first lateral side 316 and the second lateral side 318, and a fourth side 322 opposite the third side 320.

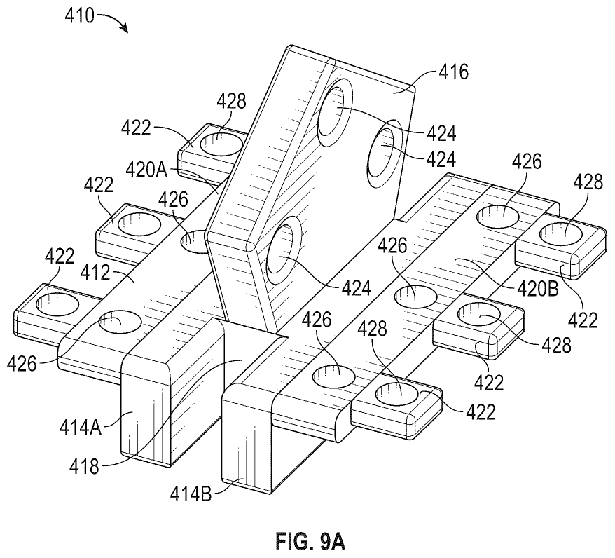

In addition, the body 312 of the spinal implant 310 may further include a second member 330 oriented in perpendicular relation relative to the first member 314. The second member 330 may be generally rectangular as shown and may define a first end 332 (shown in FIG. 8) in communication with the first member 314 along a generally central area of the fourth side 322 of the first member 314, and a second (free) end 334 opposite the first end 332. In some embodiments, the first member 314 and the second member 330 collectively define a general t-shape configuration that is intended to simulate the removed portions of a vertebral arch (not shown) subsequent to a laminectomy procedure. It should be appreciated that the body 312 of the spinal implant 310 is not limited to certain shapes and may take on various forms and dimensions so long as the spinal implant 310 accommodates the occupation of postoperative dead space, and provides other features as described herein.

Further, the spinal implant 310 may include one or more of an interconnecting arrangement 340, such as an interconnecting arrangement 340A, and an interconnecting arrangement 340B shown, for interconnecting the spinal implant 310 with one or more adjacent spinal implants similar to the spinal implant 310, such as a spinal implant 342 and a spinal implant 344. In this manner, a plurality of spinal implants such as the spinal implant 310, spinal implant 342, and the spinal implant 344, may be chained together or otherwise interconnected along a spine.

In some embodiments, the interconnecting arrangement 340A may include an arm 346A and an arm 346B defined along a first side 347 of the second member 330 to accommodate offset connections respectively with an arm 348A and an arm 348B defined along the spinal implant 342. The arm 346A and the arm 346B of the spinal implant 310 may be rotatably coupled to the arm 348A and the arm 348B of the spinal implant 342 using a pin 349 at least partially disposed through a portion of each of the arms 346A and 346B and the arms 348A and 348B. A first hinge, designated H1, may be defined along the connection of the arm 346A and the arm 346B of the spinal implant 310 and the arm 348A and the arm 348B of the spinal implant 342 via the pin 349, such that the spinal implant 310 and the spinal implant 342 may rotate relative to one another along a fixed axis of rotation.

Similarly, the interconnecting arrangement 340B may include an arm 350A and an arm 350B defined along a second side 351 (opposite the first side 347) of the second member 330 to accommodate offset connections respectively with an arm 352A and an arm 352B defined along the spinal implant 342. The arm 350A and the arm 350B of the spinal implant 310 may be rotatably coupled to the arm 352A and the arm 352B of the spinal implant 344 using a pin 353 at least partially disposed through a portion of each of the arms 350A and 350B and the arms 352A and 352B. A second hinge, designated H2, may be defined along the connection of the arm 350A and the arm 350B of the spinal implant 310 and the arm 352A and the arm 352B of the spinal implant 344 via the pin 353, such that the spinal implant 310 and the spinal implant 344 may rotate relative to one another along a fixed axis of rotation.

FIG. 8 illustrates additional detail regarding possible orientation of the arms 346A and 346B and the arms 350A and 350B of the spinal implant 310. As indicated, the arm 346A may be coupled to a surface 360 of the second member 330 proximate to the first side 347, and the arm 346B may be coupled to a surface 362 of the second member 330 opposite the surface 360, proximate to the first side 347 of the second member 330 and proximate to the second end 334 of the second member 330. Further, the arm 350A may be coupled to the surface 360 of the second member 330 proximate to the second side 351 of the second member 330, and the arm 350B may be coupled to the surface 362 of the second member 330 proximate to the second side 351 of the second member 330. In this manner, the arm 346A and the arm 346B may be oriented in parallel relation relative to one another along the first side 347 of the second member 330, and the arm 350A and the arm 350B may be oriented in parallel relation relative to one another along the second side 351 of the second member 330. In some embodiments, each of the arms 346A and 346B and the arms 350A and 350B of the spinal implant 310 are fixed in the position shown. Alternatively, the arms 346A and 346B and the arms 350A and 350B may be engaged to the spinal implant 310 in a manner that accommodates some degree of rotation of the arms 346A and 346B and the arms 350A and 350B relative to the spinal implant 310 for easier coupling to adjacent spinal implants.

Referring back to FIG. 7, the combination of the spinal implant 310 and the interconnecting arrangement 340A and/or the interconnecting arrangement 340B may effectively function akin to a cross link-type mechanism, similar to the spinal fixation construct 211, for spinal fixation. Unlike existing cross-link or pedicle screw assemblies however, the spinal implant 310 occupies dead space, and more closely resembles the removed vertebral arch. In some embodiments, the arms 346A and 346B and the arms 350A and 350B of the spinal implant 310 may be formed with a metal or other rigid or semi-rigid material, when deployed as part of and the interconnecting arrangement 340A and/or the interconnecting arrangement 340B. In addition, the first hinge H1 and the second hinge H2 defined by the interconnecting arrangement 340A and the interconnecting arrangement 340B may accommodate controlled forward bending of vertebral segments to address proximal junctional failure or adjacent segment disease.

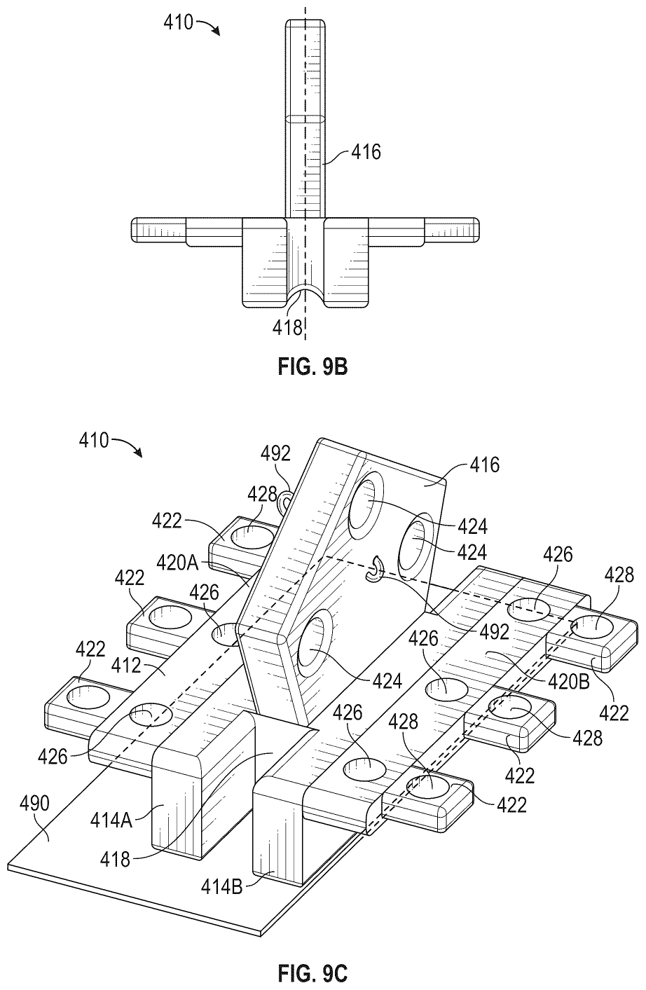

Referring to FIG. 9A, a fourth embodiment of a spinal implant 410 is shown which may be implemented along a surgical area associated with a laminectomy procedure similar to the spinal implants 110, 210, and 310 described herein. The spinal implant 410 may include a body 412 defining a first lamina support 414A, a second lamina support 414B, a plate section 416 extending outwardly between the first lamina support 414A and the second lamina support 414B, and a support bridge 418 defined between the first lamina support 414A and the second lamina support 414B. In addition, the spinal implant 410 may include a first screw shelf 420A extending from the first lamina support 414A, and a second screw shelf 420B extending from the second lamina support 414B, wherein both the first screw shelf 420A and the second screw shelf 420B may be removably coupled to at least one removable screw tab 422.

In this non-limiting embodiment, the spinous process plate section 416 further includes at least one plate section opening 424 through which the paraspinal muscles may be attached or at least partially received. Furthermore, the first screw shelf 420A, and the second screw shelf 420B, may include at least one screw shelf opening 426 for receiving a screw (not shown) or other fastening member for engaging with bone tissue or a spinal fixation construct. It is contemplated that the screw shelf openings 426 can vary in diameter to accommodate different screw sizes such that the device may be substantially secured to the engaged muscle, tissue, or other material. In addition, the removable screw tabs 422 may include respective screw tab openings 428 through which screw tab screws (not shown) may be threaded such that the surgeon may substantially secure the spinal implant 410 to a patient's bone. It is contemplated that the spinal implant 410 may include an alternative fastening device without departing from the scope of the disclosure. For example, the spinal implant 410 may be surgically tethered, fused, fixed or any combination of those, to the patient.

In this non-limiting embodiment, the first lamina support 414A is separated from the second lamina support 414B by the support bridge 418. However, it is contemplated that they may also be separated by the spinous process plate section 416. In addition, it is also contemplated that the first lamina support 414A and the second lamina support 414B may be configured to attach to each other. For example, a support bar (not shown) may be installed within the space between the first lamina support 414A and the second lamina support 414B. This support bar may be substantially rigid, or it may be configured to allow relative motion between the first lamina support 414A and the second lamina support 414B. Additionally, the first lamina support 414A and the second lamina support 414B may include curved bottom surfaces that may be capable of closely engaging with portions of the cut lamina (not shown).

In one non-limiting embodiment, the removable screw tabs 422 are configured to be detached from the first screw shelf 420A or the second screw shelf 420B such that they may accommodate varying interpeduncular distance. To illustrate, in this non-limiting embodiment, the surgeon may cut off any excess or undesired screw tabs 422 in order to provide the patient with a better implant. If the patient's spine is particularly wide, the at least one removable screw tabs 422 may be used by the surgeon to connect the device to the patient; however, if the spine is too narrow for the removable screw tabs 422 than any or all of them may be removed by the surgeon so that the connection can be made directly with the first screw shelf 420A and the second screw shelf 420B. In addition, the removable screw tabs 422 may have a reduced thickness compared to the first screw shelf 420A or the second screw shelf 420B such that the surgeon may quickly and easily cut off the screw tab or tabs 422 that are not needed, while still sufficiently thick enough to substantially achieve a secure engagement of the muscle, tissue, or other material.

Furthermore, it is contemplated that the at least one of the removable screw tabs 422 may vary in number and in distance between each tab and perform substantially the same function without departing from the scope of the disclosure. In one non-limiting example, the body of the removable screw tabs 422 may further include another removable screw tab (not shown) extending outwardly therefrom from, with a profile that generally matches the dimensions of the screw tabs 422. In this case, another removable screw tab may be separated by a scored section (not shown) that has a substantially reduced material thickness in comparison to the screw tabs 422 such that a surgeon may quickly and easily separate one tab from another. It is also contemplated that the at least one removable screw tab 422 may comprise any known or suitable alternative to size and shape, location, configuration, etc. without departing from the scope of the disclosure. It is further appreciated that any known or suitable alternative configuration of the at least one removable screw tab 422 could be employed, such as multi-level removable screw tabs, wherein the one or both of the screw shelves may extend along a first vertical axis such that the screw shelf can be configured to attach to multiple screw tabs each stacked upon one another such that the screw tab screws may extend through multiple screw tab openings and can be threadably received by the screw tab openings. It is also contemplated that the at least one removable screw tab 422 may extend from the first screw shelf 420A and second screw shelf 420B from different angles. In addition, the at least one removable screw tab 422 may also be arranged in a shaft-loop configuration.

In another non-limiting embodiment, the spinal implant 410 may further include an anchor jaw assembly (not shown). The anchor jaw assembly may include a rectangular support component (not shown) that may extend outward from the anterior or posterior surface of the support bridge 418, first lamina support 414A, second lamina support 414B, first screw shelf 420A, second screw shelf 420B, or any combination of these components, with the support component being adjustably secured to an anchor jaw (not shown). The anchor jaw may include a dual-prong configuration that extends outward from the support component and may comprise a first elongated prong of the anchor jaw, a second elongated prong of the anchor jaw, and a plurality of jaw openings positioned on both the first prong, and the second prong. In this manner, a screw, rail, pin, or other suitable fastening device may extend through the first prong of the anchor jaw, the patients natural spinous process, and the second prong of the anchor jaw so that a surgeon may fix the spinal implant 410 to inferior and superior adjacent spinous processes, additional spinous process implants, or both, as a means to correct multilevel laminar defects or other relevant conditions. The anchor jaw may integrate with these features in other methods as well. For example, in one non-limiting embodiment, the anchor jaw may also be designed as a rotatable clamp structure (not shown) that may secure the assembly to a feature by compressing the prongs, against the feature, and wherein the rotatable clamp structure is rotatable about an anchor locking position disposed between the rectangular support component and the clamp structure so that the assembly may accommodate for variations in the curvature of the patient's spine.

In another non-limiting embodiment, shown in FIG. 9B, the support bridge 418 may include a curved bottom surface, which may be configured such that it curves substantially inwards towards the spinous process plate section 416. In this non-limiting embodiment, the curved bottom surface provides the device with a decompression zone so that it may relieve pressure caused by spinal cord abnormalities without compromising the protection allotted by the device. Additionally, the support bridge 418 may have a substantially flat surface, sloped surface, or a combination of flat, sloped, and curved surfaces. For example, the support bridge 418 may have a bottom surface that is gradually sloped from anterior end to posterior end such that it forms a modest angle in one embodiment. Further, the curved bottom surface could have a plurality of protrusions extending away from the bottom surface in any number of configurations such that they may provide substantial surface contact with the patient to allow for better a better fit while still providing the benefits of the decompression zone.

As further shown in FIG. 9A, the plate section openings 424 can be seen at the anterior, posterior, and top edge of the spinous process plate section 416. The varying height arrangement of these plate section openings 424 provides the surgeon with an array of options for connecting the muscle, tissues, or other materials along the spinal implant 410. It is also contemplated that the number of plate section openings 424, and the size of the plate section openings 424 may vary without departing from the scope of the present disclosure.

In addition, in another non-limiting embodiment, the spinous process plate section 416 may include a plurality of engagement members 492 (shown in FIG. 9C), which may include plate section loops extending outward from the spinous process plate section 416 which may also provide the surgeon with sufficient coverage and attachment options. Arranged across the spinous process plate section 416, these outwardly facing loops may be constructed at different orientations and still achieve secure engagement of the muscle, tissue, or other material to the spinal implant 410. Additionally, it is also contemplated that the loops could be designed as hooks and achieve substantially the same function without departing from the scope of the disclosure. As an example, the plurality of plate section loops may be designed such that the component material curves around and crosses itself to form a complete loop. However, in this non-limiting embodiment, the loop may not cross itself such that it forms a hook and still allows for the purpose of sewing the paraspinal muscles to the spinal implant 410.

It is further contemplated that a person with ordinary skill in the art would understand that the at least one of the screw tab openings 428, the plate section openings 424, and the screw shelf openings 426 may have different shapes depending on the surgeon's needs. For example, the plate section openings 424 may have a square shape or an oblong shape and serve substantially the same purpose without departing from the scope of the disclosure. In addition, although not depicted, any of the openings may have a textured or threaded surface such that they are configured to receive a suitable fastening device.

In some embodiments, the first lamina support 414A and the second lamina support 414B can generally be shaped into any substantially structurally compliant form. For example, the first lamina support 414A may comprise a straight rectangular portion, a straight rectangular portion and a portion angled outward from the support bridge 418, a straight cylindrical portion, or any combination of these or forms. In one non-limiting embodiment, the first lamina support 414A and the second lamina support 414B may have substantially different lengths than depicted. Alternatively, the first lamina support 414A. In one non-limiting embodiment, the spinal implant 410 may be designed without either of the first lamina support 414A and the second lamina support 414B. For example, the spinous process plate section 416 may be configured to attach directly to the first screw shelf 420A, second screw shelf 420B, and the support bridge 418.

Additionally, the spinous process plate section 416 is depicted having a pentagon-shaped dimension, but it should be understood by a person of ordinary skill in the art that the spinous process plate section 416 may be comprised in other shapes and forms without departing from the scope of the disclosure. For example, it is contemplated that the spinous process plate section 416 may have a substantially rounded dimension instead of the pentagonal dimension depicted in the figures. In addition, the spinous process plate section 416 could be designed to imitate the natural appearance of a person's spinous process.

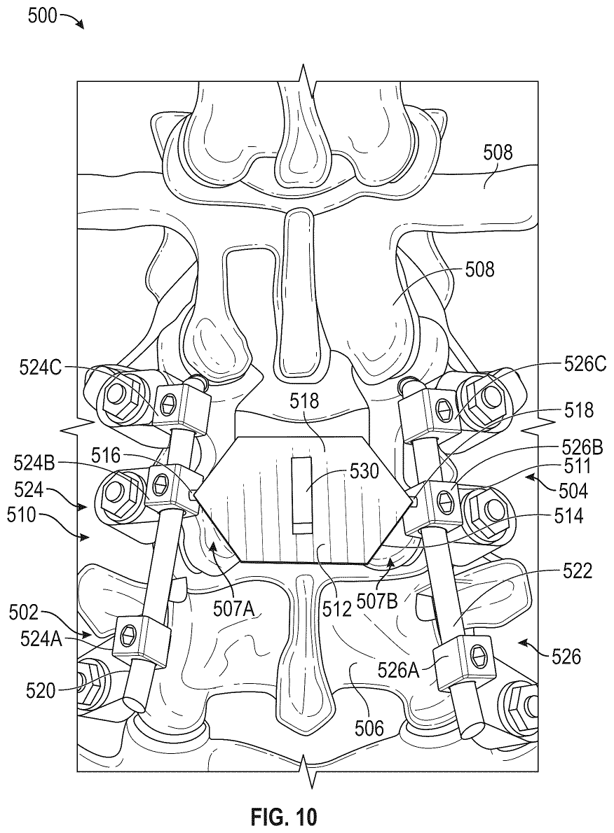

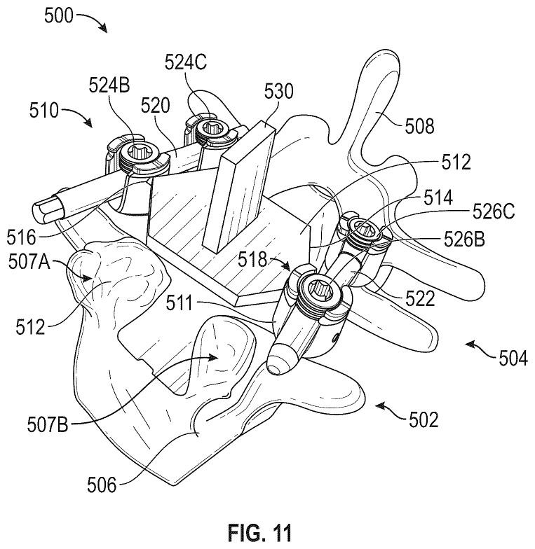

Referring to FIGS. 10-11, a fifth embodiment of a spinal implant 510 according to a system 500, may be positioned along a first vertebral segment 504 of a spine 502 between a second vertebral segment 506 and a third vertebral segment 508 in the manner indicated, and may generally be mounted to or at least oriented along cut lamina portions 507A and 507B of the first vertebral segment 504 in order to occupy dead space resulting from a laminectomy procedure applied to the first vertebral segment 504. The spinal implant 510 may provide various postoperative advantages as described herein.

As further shown, the spinal implant 510 may be integrated with a spinal fixation construct 511 comprising one or more pedicle screws, rods, or other spinal fixation hardware that is coupled to the spine 502. In the embodiment shown, the spinal fixation construct 511 may include any cross link system for spinal fixation as would be understood by one skilled in the art. The spinal implant 510 may be engaged along the first vertebral segment 504 prior to application of the spinal fixation construct 511, together with application of the spinal fixation construct 511, or otherwise, as further described herein.

The spinal implant 510 may include a body 512 defining a first member 514. The first member 514 may be generally formed with dimensions suitable for engagement along portions of the first vertebral segment 504 such as the cut lamina portions 507A/507B and may be generally formed with dimensions suitable for connections to portions of the spinal fixation construct 511, as further described herein. In some embodiments, the first member 514 may further generally comprise a six-sided shape configuration, and may be substantially planar. In addition, the first member 514 of the body 512 may define a first lateral corner 516, and a second lateral corner 518 opposite the first lateral corner 516 as shown.

As further shown, the spinal implant 510 may be integrated with the spinal fixation construct 211. The spinal fixation construct 511 may include a first rod 520 and a second rod 522 oriented in parallel orientation relative to one another and positioned along the spine 502 as indicated. The spinal fixation construct 511 may further include one or more connection portions 524, illustrated as 524A (not shown in FIG. 11), 524B, and 524C, defined along the first rod 520, and one or more connection portions 526, illustrated as 526A (not shown in FIG. 11), 526B, and 526C, defined along the first rod 520, shown as 524A, 524B, and 524C, defined along the second rod 522. In some embodiments, the first lateral corner 516 may be connected or otherwise engaged to the connection portion 524B, and the second lateral corner 518 may be connected or otherwise engaged to the connection portion 526B as shown. In this manner, the spinal implant 510 may be suspended over the cut lamina portions 507A/507B of the first vertebral segment 504, and may function akin to a cross-link, being similarly integrated with the spinal fixation construct 511. The combination of the spinal fixation construct 511 and the spinal implant 510 re-occupies much of the dead space resulting from a laminectomy procedure applied to the first vertebral segment 504, and the spinal fixation construct 511 may assist to secure the spinal implant 510 in place relative to the spine 502 as indicated. In some embodiments, other connection portions 524 and 526 may be engaged with other spinal implants (not shown) and/or or cross-links (not shown).

In addition, the body 512 of the spinal implant 510 may further include a second member 530 oriented in substantially perpendicular relation relative to the first member 514. The second member 530 may be generally rectangular as shown and may extend from a generally central area of the first member 514, as shown. In some embodiments, the first member 514 and the second member 530 collectively define a general t-shape configuration (from a side view) that is intended to simulate the removed portions of a vertebral arch (not shown) along the first vertebral segment 504 subsequent to a laminectomy procedure.

In one embodiment, the spinal implant 510 is rigidly affixed to the spinal fixation construct 511 in the position shown in order to add rigidity to the entirety of the spinal fixation construct 511. In addition, the spinal implant 510 at least partially fills postoperative dead space along the cut lamina portions 507A/507B, and provides a suitable object for engagement to paraspinal muscles. As such, the spinal implant 510 provides a novel improvement to existing cross-link members, as the spinal implant 510 at least somewhat resembles the removed vertebral arch (not shown) while also providing additional functionality for spinal fixation and recovery.

It should be appreciated that the body 512 of the spinal implant 510 is not limited to certain shapes and may take on various forms and dimensions so long as the spinal implant 510 accommodates the occupation of postoperative dead space and the depicted connections to the spinal fixation construct 511, and provides other features as described herein. The body 512 may be manufactured or comprised of any number of suitable sterilizible and/or biocompatible materials, such as metal, polymer, alloy, biodegradable composite, bioactive material, resin, ceramic, or any combinations of the same. In addition, the surface of the spinal implant 510 may be coated with any number of suitable materials.

The embodiments of the spinal implants 110, 210, 310, 410, and 510 described herein may include various sub features or variations. For example, the spinal implants 110, 210, 310, 410, and 510 may include smooth surfaces, and/or may include surface features such as ridges, bumps, protrusions, channels or any combination of these elements without departing from the scope of the disclosure. These features may be advantageous for interacting or diverting the flow of liquid over the device during surgery. In addition, these features may be dispersed across the device in any known configuration to the preference of the user. Furthermore, the components of the spinal implants 110, 210, 310, 410, and 510 may be manufactured or comprised of any number of suitable sterilizible materials, such as metal, polymer, alloy, biodegradable composite, bioactive material, resin, ceramic, or any combination of these. In addition, the surface of the device may be coated with any number of suitable materials. Any of the spinal implants 110, 210, 310, 410, and 510 may be manufactured by connecting various discrete components, or by unitary construction.

Moreover, any of the spinal implants 110, 210, 310, 410, and 510 may be manufactured such that any interior part of the device, or the entire interior, is hollow. In this manner, any of the spinal implants 110, 210, 310, 410, and 510 can be partially or completely filled with antibiotic material, solutions, bioactive materials, or any combinations of the same. In addition, in a further implementation, any hollow interior of the spinal implants 110, 210, 310, 410, and 510 may comprise solid components as well. For example, in one non-limiting embodiment, the hollow interior may comprise at least one column shaped honeycomb structure. These columns may be arranged in such a manner that they cross-link with one another or alternatively, they may be arranged to be substantially parallel with one another along a face of the interior of the spinal implants 110, 210, 310, 410, and 510 both of which may provide the spinal implants 110, 210, 310, 410, and 510 with significant structural support. The honeycomb structures may be coated with any number of organic or inorganic substances, including catalysts, binders or any combination of these. Furthermore, the honeycomb structures could also comprise a plurality of pores dispersed along the length of the honeycomb structures.

For cases that require spinal fixation hardware, any of the spinal implants 110, 210, 310, 410, and 510 may include a bottom member or mounting rack (shown in FIG. 9C as 490) that may be defined along the bottom surface of the spinal implants 110, 210, 310, 410, and 510. In this manner, the bottom member may be arranged such that it may be slidably connected to the spinal implants 110, 210, 310, 410, and 510 or other objects around the surgical site via a connection mechanism. Some non-limiting embodiments of the connection mechanism include mechanical components that lock together, but are be readily separated by pressing and pulling so that the bottom member slides along a track. In this non-limiting embodiment, it is conceivable that the connection mechanism includes a series of locking positions dispersed along the track in order to adapt to differing interpeduncular lengths. It is contemplated that the bottom member may be slidably coupled to the spinal implants 110, 210, 310, 410, and 510 by non-mechanical methods as well, or alternatively, it may be rigidly connected to the spinal implants 110, 210, 310, 410, and 510.

In some embodiments, the spinal implants 110, 210, 310, 410, and 510 may include one or more hooks (shown in FIG. 9C as engagement members 492) to accommodate engagement of the spinal implants 110, 210, 310, 410, and 510 to spinal fixation rods (shown e.g., in FIG. 5A) or bodily tissue. However, adhesives, pins, rails, clamps, or any other suitable method for connecting two components together are may be employed. Additionally, it is contemplated that in a similar non-limiting embodiment, the hooks could be configured to attach to pedicle screws. For example, the hooks could be designed such that one or more hook like devices cooperate with the frame of the screw so that the spinal implants 110, 210, 310, 410, and 510 may be stabilized on the patient's body.

In addition, the spinal implants 110, 210, 310, 410, and 510 are depicted as one component. However, it is contemplated that the spinal implants 110, 210, 310, 410, and 510 can be constructed out of multiple components; for example, the plate section 416 of the spinal implant 410 could be comprised of two separable pieces. Further, any of the spinal implants 110, 210, 310, 410, and 510 may be printed at least partially using a three-dimensional printing device, using predetermined materials suitable for replicating bone tissue. The dimensions of the spinal implants 110, 210, 310, 410, and 510 may be determined based on a medical image of an actual patient or cadaver to generate the spinal implants 110, 210, 310, 410, and 510 with desired anatomical properties.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system, comprising:
a first spinal implant including a body comprising a biocompatible material, the body defining:
a first member configured for mechanical engagement along a partially excised vertebral segment, the first member being substantially planar and defining a first surface and a second surface;
a spinous process member extending from the first surface of the first member, the first member and the spinous process member collectively defining a T-shape configuration; and
a first lamina support member and a second lamina support member extending from the second surface of the first member, the first lamina support member and the second lamina support member being in parallel relation relative to one another and the spinous process member;
wherein the first spinal implant is configured for at least partially occupying dead space along a surgical site such that the first lamina support member and the second lamina support member occupy dead space along the surgical site previously occupied by excised lamina.

2. The system of claim 1, further comprising a support bridge defined along the second surface of the first member between the first lamina support member and the second lamina support member, the support bridge including a curved bottom surface that establishes a decompression zone aligning with a spinal cord.

3. The system of claim 1, the first member further comprising:
a first screw shelf extending laterally from the first lamina support member and a second screw shelf extending laterally from the second lamina support member.

4. The system of claim 3, wherein the first screw shelf and the second screw shelf each define at least one opening for receipt of a fastening member.

5. The system of claim 3, further comprising at least one removable screw tab positioned lateral to the first screw shelf or the second screw shelf and defining at least one opening for receipt of a fastening member.

6. The system of claim 1, the spinous process member replacing an excised spinous process of the partially excised vertebral segment.

7. A spinal implant, comprising:
a body including:
a first lamina support member extending along a first direction and configured for mechanical engagement along a partially excised vertebral segment;
a second lamina support member extending in parallel relation to the first lamina support member; and
a support bridge defined between the first lamina support member and the second lamina support member;
wherein the body is at least partially formed with bone grafting substitutes to simulate bone tissue such that the body is at least partially porous and configured for receiving bone tissue over a predetermined amount of time, and wherein the first lamina support member and the second lamina support member are configured for occupying dead space along a surgical site previously occupied by excised lamina.

8. The spinal implant of claim 7, further comprising:
a spinous process member positioned in parallel relative to the first lamina support member and the second lamina support member along a second direction opposite the first direction, the spinous process member replacing an excised spinous process of the partially excised vertebral segment.

9. The spinal implant of claim 7, further comprising a support bridge positioned between the first lamina support member and the second lamina support member, the support bridge defining a curved bottom surface that establishes a decompression zone aligning with a spinal cord.

10. The spinal implant of claim 7, further comprising:
a first screw shelf extending laterally from the first lamina support member and a second screw shelf extending laterally from the second lamina support member.

11. The spinal implant of claim 10, wherein the first screw shelf and the second screw shelf each define an opening for receipt of a fastening member.

12. The spinal implant of claim 10, further comprising at least one removable screw tab positioned lateral to the first screw shelf or the second screw shelf and defining an opening for receipt of a fastening member.
